(19) United States
(12) Patent Application Publication
(10) Pub. No.: US 2026/0133424 A1
(43) Pub. Date: May 14, 2026

(54) REDUCTION OF JITTER IN VIRTUAL PRESENTATION

(71) Applicant: AUGMEDICS LTD., Yokneam Illit (IL)

(72) Inventor: Asaf Asaban, Haifa (IL)

(21) Appl. No.: 18/857,558

(22) PCT Filed: Apr. 20, 2023
(86) PCT No.: PCT/IB2023/054057

§ 371 Date: Oct. 17, 2024

Related U.S. Application Data
(60) Provisional application No. 63/333,127, filed on Apr. 21, 2022.
(60) Provisional application No. 63/429,177, filed on Dec. 1, 2022.

(51) Int. Cl. G02B 27/01; A61B 34/20; A61B 90/00; A61B 90/50; G06T 5/20; G06T 7/00; G06T 7/73; G06T 19/00

(52) U.S. Cl. CPC G02B 27/0172; A61B 34/20; G06T 5/20; G06T 7/0012; G06T 7/73; G06T 19/006; A61B 2034/2055; A61B 2090/373; A61B 2090/502; G02B 2027/0138; G02B 2027/0178; G06T 2207/10048; G06T 2207/30012; G06T 2207/30204; G06T 2210/41

(57) ABSTRACT

A computer-implemented method, consisting of acquiring a first image of reflective elements of an identifying marker positioned in proximity to a region of interest of a subject and calculating a first position of the identifying marker in response to the first image. The method continues with acquiring, subsequent to acquiring the first image, a second image of the reflective elements and calculating a second position of the identifying marker in response to the second image. Subsequent to acquiring the second image, the method continues with acquiring further images of the reflective elements and calculating respective further positions of the identifying marker in response to the further images. The further positions are filtered in response to a difference between the second position and the first position. In response to the filtered further positions, an image of an entity associated with the identifying marker is presented on a display.

### CROSS-REFERENCE TO RELATED APPLICATIONS

This application claims the benefit of U.S. Provisional Ser. No. 63/333,127, filed Apr. 21, 2022 and U.S. Provisional Ser. No. 63/429,177 , filed Dec. 1, 2022; the entire content of each of which is incorporated herein by reference.

### FIELD OF THE DISCLOSURE

This disclosure relates generally to virtual image presentation, including for example, to reduction of jitter in virtual images presented on, or in conjunction with, an image-guided medical and/or diagnostic procedure, such as an augmented reality image-guided medical procedure, non-surgical procedure and/or diagnostic procedure.

### BACKGROUND

Virtual presentation may be used advantageously to assist visualization, e.g., of a medical procedure, by displaying in a correctly aligned manner, elements of the procedure that are normally hidden from direct view. For example, during a procedure performed by a surgeon on the spine of a patient, an image comprising an aligned combination of a stored image of the spine with a virtual image of a tool used by the surgeon to operate on the spine, may be presented to the surgeon, while the image includes at least a portion of the spine which is not visible to the surgeon. If a head-mounted display system is used, then an augmented reality image may be displayed by aligning the image with the scene visible through the display of the head-mounted display system.

### SUMMARY

In accordance with several embodiments, systems and methods are for improving presentation of virtual images on a display are described herein. The virtual images may include images of portions of a patient anatomy and/or virtual images of physical tools, instruments, or devices (such as insertion tools or instruments, implants, screws, hardware, diagnostic tools or instruments, surgical tools or instruments, treatment tools or instruments, etc.) being tracked by a tracking system. The display may be an augmented reality display, virtual reality display, or mixed reality display of a hands-free (e.g., head-mounted) display device or system (e.g., glasses, goggles, eyewear, over-the-head unit) or other augmented reality display. The display may be an augmented reality display, virtual reality display, or mixed reality display of a display device that is not head-mounted (e.g., a portal or tablet or monitor). The display may be a standalone, separate display. The tracking system may include one or more imaging devices (e.g., one or more cameras, such as one or more infrared cameras and/or red-green-blue (RGB) cameras, optical imaging devices, ultrasound imaging devices, thermal imaging devices, radar imaging devices, or other imaging devices) configured to capture still images and/or video images or one or more reading devices. The images may be 2D images, 3D images, or 4D images. For infrared imaging devices, the images may include reflections received from reflective markers coupled to the physical instruments, tools or devices. The tracking system may additionally or alternatively include one or more non-imaging devices for tracking locations of devices, such as radiofrequency identification (RFID) readers, near field communication (NFC) readers, optical code readers. detectors or scanners, Bluetooth sensors, position sensors, electromagnetic sensors, or other wireless sensing devices.

In accordance with several embodiments, the tracking system introduces noise in the display (e.g., as a result of motion of the instruments, tools or devices being tracked). The noise may result in a jitter effect (e.g., flickering) in the display of the virtual images that may affect the user experience. The jitter may result in a misalignment between the physical instrument, tool or device and the actual, real environment (and/or the virtual image of the instrument, tool or device) that are being viewed by an operator (e.g., a physician or other clinical professional). The jitter may also affect performance of one or more processors or may cause loss of transmitted data between connected devices over a communications network.

In accordance with several embodiments, it may be advantageous to reduce jitter in order to improve the user experience and the confidence in the accuracy of the display.

In accordance with several embodiments, reduction of jitter does not noticeably or significantly increase latency between movement of the physical device, tool, or instrument and the display of the virtual image of the device, tool, or instrument on the display. For example, jitter reduction techniques described herein may involve application of filtering only when the scene being imaged is determined to be dynamic or changing enough to warrant jitter reduction techniques but not when the scene being imaged is relatively static. The determinations may involve one or more thresholds that are applied to the images and evaluated to make the determinations.

The jitter reduction techniques may include application or activation of one or more filters (e.g., low pass filters designed to filter out high frequency noise). The filters may include cutoff frequencies.

The thresholds may involve calculation of differences between positions of locations of at least a portion (e.g., centroid) of the physical tool, instrument or device) being tracked (e.g., based on tracking of a marker coupled thereto).

In accordance with several embodiments, if values are determined to be satisfy criteria associated with one or more thresholds (e.g., are above or below a threshold value), then jitter reduction techniques (e.g., filtering) may be applied. If the one or more criteria are not satisfied, then jitter reduction techniques may not be applied, which may reduce overall latency in the user experience.

In accordance with several embodiments, a system for reducing jitter in display presentations during image-guided medical procedures comprises or consists essentially of an identifying marker having reflective elements thereon configured to be coupled to an instrument or tool configured for use in a medical procedure and a wearable device including a camera configured to acquire images of the reflective elements of the identifying marker and a display configured to display a virtual augmented reality image of at least a portion of the instrument or tool while a wearer of the wearable device can still see through the display. The wearable device also includes one or more processors.

The one or more processors may be configured to (e.g., upon execution of stored program instructions on one or more non-transitory computer-readable media): operate the camera to acquire a first image of the reflective elements and to calculate a first position of the identifying marker in response to the first image; operate the camera, subsequent to acquiring the first image, to acquire a second image of the reflective elements and to calculate a second position of the identifying marker in response to the second image; operate the camera, subsequent to acquiring the second image, to acquire further images of the reflective elements and to calculate respective further positions of the identifying marker in response to the further images; filter the further positions in response to a difference between the second position and the first position; and in response to the filtered further positions, generate the virtual augmented reality image of at least the portion of the instrument or tool on the display of the wearable device.

The wearable device may include a head-mounted device, a non-head-mounted device but hands-free device, or a combination of head-mounted and non-head-mounted components. For example, the head-mounted device may be a pair of glasses or goggles or other eyewear. The head-mounted device may be an over-the-head mounted device.

In some embodiments, the first and the second positions are two-dimensional positions measured in pixels, and the difference between the second position and the first position is a velocity from the first position to the second position measured in pixels per second.

In some embodiments, the first and the second positions are three-dimensional positions, and the difference between the second position and the first position is a velocity from the first position to the second position.

In some embodiments, the camera is an infrared camera.

In some embodiments, when the difference is not greater than a threshold, the filtering comprises applying first filtering (e.g., a first low-pass filter) with a first cutoff frequency, and when the difference is greater than the threshold, the filtering comprises applying second filtering (e.g., a second low-pass filter) with a second cutoff frequency greater than the first cutoff frequency.

The second cutoff frequency may be a frequency having a value between 3 Hz and 10 Hz (e.g., between 4 Hz and 7 Hz). The first cutoff frequency may be a frequency having a value between 0 Hz and 3 Hz (e.g., between 0 Hz and 2 Hz).

In some embodiments, one or both of the cutoff frequencies are preset frequencies of a stored preset value. In some embodiments, one or both of the cutoff frequencies are automatically adjusted over time by the one or more processors.

In some embodiments, the first low-pass filter and the second low-pass filter are infinite impulse response filters (e.g., a first order infinite impulse response filter). However, other digital filters may be used in some embodiments.

In some embodiments, the threshold is a stored preset value. The threshold value may be provided by a user or automatically calculated by the one or more processors.

In some embodiments, the system further includes a second identifying marker having a local vector indicative of a region of interest associated with the medical procedure. The second identifying marker may be configured to be coupled to a portion of a patient at or near the region of interest. In some embodiments, the second identifying marker includes reflective elements configured to be imaged by the camera so as to facilitate determination of a location of the second identifying marker that is used by the one or more processors to calculate the first position, the second position, and the further positions. For example, the two identifying markers may be registered or calibrated with each other to facilitate position calculations in a particular field of reference.

In accordance with several embodiments, a method for reducing jitter in display presentations during image-guided surgery includes acquiring a first image of reflective elements of a first identifying marker coupled to an instrument or tool configured for use in a medical procedure (e.g., spinal surgical procedure or other surgical or non-surgical procedure, such as a therapeutic and/or diagnostic procedure) and of reflective elements of a second identifying marker coupled to a patient and positioned in proximity to a region of interest of the patient associated with the medical procedure. The method also includes calculating a first position of the first identifying marker in response to the first image with reference to a determined first location of the second identifying marker. The method further includes acquiring, subsequent to acquiring the first image, a second image of the reflective elements of the first identifying marker and the second identifying marker and calculating a second position of the first identifying marker in response to the second image. The method also includes acquiring, subsequent to acquiring the second image, further images of the reflective elements of the first identifying marker and the second identifying marker and calculating respective further positions of the first identifying marker in response to the further images. The method further includes filtering the further positions in response to a difference between the second position and the first position. The method also includes, in response to the filtered further positions, generating images of at least a portion of the instrument or tool for output on a wearable display to facilitate performance of the image-guided surgery by a wearer of the wearable display.

In some embodiments, the method is performed by one or more processors on the wearable display. The wearable display may be on a head-mounted device, a non-head-mounted device or a combination of both head-mounted and non-head-mounted components. The wearable display may be glasses, goggles, or other form of eyewear.

The wearable display may be a direct see-through display. The images may include he virtual augmented reality images of at least the portion of the instrument or tool.

In some embodiments, the second identifying marker is a marker having a local vector indicative of the region of interest.

In some embodiments, when the difference is not greater than a threshold value, the filtering applying filtering (e.g., applying a first low-pass filter) with a first cutoff frequency, and when the difference is greater than the threshold value, the filtering comprises applying filtering (e.g., applying a second low-pass filter) with a second cutoff frequency greater than the first cutoff frequency.

The second cutoff frequency may be a frequency having a value between 3 Hz and 10 Hz (e.g., between 4 Hz and 7 Hz). The first cutoff frequency may be a frequency having a value between 0 Hz and 3 Hz (e.g., between 0 Hz and 2 Hz).

In some embodiments, one or both of the cutoff frequencies are preset frequencies of a stored preset value. In some embodiments, one or both of the cutoff frequencies are automatically adjusted over time by the one or more processors.

In some embodiments, the first low-pass filter and the second low-pass filter are infinite impulse response filters (e.g., a first order infinite impulse response filter). However, other digital filters may be used in some embodiments (e.g., finite impulse response filters, hiss filters, second-order filters, etc.).

In some embodiments, the method further includes diagnosing and/or treating a medical condition, the medical condition including, but not limited to, one or more of the following: back pain, spinal deformity, degeneration of the spine or other bone or joints, spinal stenosis, disc herniation, joint inflammation, joint damage, ligament or tendon ruptures or tears.

In accordance with some embodiments, a computer implemented method includes acquiring a first image of reflective elements of an identifying marker positioned in proximity to a region of interest of a subject and calculating a first position of the identifying marker in response to the first image; acquiring, subsequent to acquiring the first image, a second image of the reflective elements and calculating a second position of the identifying marker in response to the second image; acquiring, subsequent to acquiring the second image, further images of the reflective elements and calculating respective further positions of the identifying marker in response to the further images; filtering the further positions in response to a difference between the second position and the first position; and in response to the filtered further positions, presenting an image of an entity associated with the identifying marker on a display.

In some embodiments, the entity includes a tool used in a medical procedure performed on the subject, and the identifying marker includes a tool marker attached to the tool.

In some embodiments, the entity includes the region of interest of the subject, and the identifying marker includes a marker having a local vector indicative of the region of interest.

In some embodiments, when the difference is not greater than a preset threshold, the filtering includes applying a first low-pass filter with a first preset cutoff frequency, and when the difference is greater than the preset threshold, the filtering includes applying a second low-pass filter with a second preset cutoff frequency greater than the first cutoff frequency.

The second cutoff frequency may include a value between 3 Hz and 10 Hz (such as between 4 Hz and 7 Hz, between 3 Hz and 6 Hz, between 5 Hz and 10 Hz, overlapping ranges thereof, or any value within the recited ranges, such as 3 Hz, 3.5 Hz, 4 Hz, 4.5 Hz, 5 Hz, 5.5 Hz, 6 Hz, 6.5 Hz, 7 Hz, 7.5 Hz, 8 Hz, 8.5 Hz, 9 Hz, 9.5 Hz and 10 Hz).

The first cutoff frequency may include a value between 0 Hz and 4 Hz (such as between 0 and 3 Hz, between 0.5 and 2 Hz, between 1 Hz and 3 Hz, between 2 Hz and 4 Hz, overlapping ranges thereof, or any value within the recited ranges, such as 0 Hz, 0.5 Hz, 1 Hz, 1.5 Hz, 2 Hz, 2.5 Hz, 3 Hz, 3.5 Hz, 4 Hz).

In some embodiments, the first and the second positions are two-dimensional positions measured in pixels, and the difference between the second position and the first position includes a velocity from the first position to the second position measured in pixels per second or in mm/sec.

In some embodiments, the first and the second positions are three-dimensional positions, and the difference between the second position and the first position includes a velocity from the first position to the second position.

In some embodiments, the display is a display of a head-mounted device, including but not limited to glasses, googles, spectacles, visors, monocle, other eyewear, or over-the-head headset. In some embodiments, the head-mounted displays are not used or used together with stand-alone displays, such as monitors, portable devices, tablets, etc. The display may be a hands-free display such that the operator does not need to hold the display.

The head-mounted device may alternatively be a wearable device on a body part other than the head (e.g., a non-head-mounted device). The head-mounted device may be substituted with an alternative hands-free device that is not worn by the operator, such as a portal, monitor or tablet. The display may be a head-up display or heads-up display.

In some embodiments, the acquiring is performed by an infrared image-capturing device.

In various embodiments, the method is performed by one or more processors executing instructions stored on one or more non-transitory computer-readable storage media.

In accordance with several embodiments, a system or apparatus includes an identifying marker, having reflective elements thereon, configured to be positioned in proximity to a region of interest of a subject; a head-mounted display including a camera and a display; and one or more processors. The one or more processors are configured to (e.g., upon execution of program instructions stored on one or more non-transitory computer-readable storage media): operate the camera to acquire a first image of the reflective elements and to calculate a first position of the identifying marker in response to the first image; operate the camera, subsequent to acquiring the first image, to acquire a second image of the reflective elements and to calculate a second position of the identifying marker in response to the second image; operate the camera, subsequent to acquiring the second image, to acquire further images of the reflective elements and to calculate respective further positions of the identifying marker in response to the further images; filter the further positions in response to a difference between the second position and the first position; and in response to the filtered further positions, present an image of an entity associated with the identifying marker on the display.

In an embodiment, the element is a medical tool.

In some embodiments, the element includes an identifying marker and the first image, the second image and the further images include the identifying marker.

The identifying marker may include one or more reflective elements, and the first image, the second image and the further images may include at least a portion of the one or more reflective elements.

In some embodiments, the entity is a tool used in a medical procedure performed on the subject, and the identifying marker is a tool marker attached to the tool.

In some embodiments, the entity includes the region of interest of the subject, and the identifying marker comprises a marker having a local vector indicative of the region of interest.

In various embodiments, the head-mounted device comprises a pair of glasses, a pair of spectacles, a pair of goggles, other eyewear, or an over-the-head mounted device. In some embodiments, the camera is an infrared camera.

In accordance with several embodiments, a computer implemented method includes acquiring a first image of an element positioned in a Region Of Interest (ROI) of a subject anatomy or in proximity to the ROI and calculating a first position of the element in response to the first image; acquiring, subsequent to acquiring the first image, a second image of the element and calculating a second position of the element in response to the second image; acquiring, subsequent to acquiring the second image, further images of the element and calculating respective further positions of the element in response to the further images; filtering the further positions in response to a difference between the second position and the first position; and in response to the filtered further positions, presenting an image of the element on a display.

In accordance with several embodiments, a computer-implemented method includes acquiring a first image of an element positioned in a region of interest (ROI) of a subject anatomy or in proximity to the ROI and calculating a first position of the element in response to the first image; acquiring, subsequent to acquiring the first image, a second image of the element and calculating a second position of the element in response to the second image; acquiring, subsequent to acquiring the second image, further images of the element and calculating respective further positions of the element in response to the further images; filtering the further positions in response to a difference between the second position and the first position; and in response to the filtered further positions, generating a virtual image of the element for output on a display.

In various implementations, the element is a medical tool, a surgical tool, or an implant. In some implementations, the element includes an identifying marker and the first image, the second image and the further images include the identifying marker.

In some implementations, the acquiring is performed by an infrared camera or sensor. In some implementations, the identifying marker includes one or more reflective elements, and the first image, the second image and the further images include at least a portion of the one or more reflective elements.

Calculating the first position of the element in response to the first image and calculating the second position of the element in response to the second image may include calculating a position of the identifying marker.

In some implementations, filtering the further positions in response to the difference between the second position and the first position includes low pass filtering with a first cutoff frequency when the difference between the second position and the first position is greater than a threshold value and low pass filtering with a second cutoff frequency when the difference is less than or equal to a threshold value.

The first cutoff frequency may be between 4 Hz and 7 Hz and the second cutoff frequency may be between 0 Hz and 3 Hz.

In some implementations, the region of interest is a portion of a spine, shoulder, knee, hip, ankle, leg, foot, arm, brain, torso, mouth, or other anatomical region.

The display may be a display of a head-mounted display device, a non-head-mounted display device or a combination of head-mounted display device elements and non-head-mounted display device elements.

In accordance with several embodiments, an apparatus includes or consists essentially of a hands-free device (e.g., wearable device such as a head-mounted device) including an imaging means and a display and one or more processors. The imaging means is configured to acquire images of an element of a medical device and the display is configured to display a virtual augmented reality image of the medical device while a wearer can still see through the display. The one or more processors are configured to (e.g., upon execution of program instructions stored on one or more non-transitory computer-readable storage media): acquire a first image of the element and calculate a first position of the element in response to the first image; acquire, subsequent to acquiring the first image, a second image of the element and calculate a second position of the element in response to the second image; acquire, subsequent to acquiring the second image, further images of the element and calculate respective further positions of the element in response to the further images; filter the further positions in response to a difference between the second position and the first position; and in response to the filtered further positions, generate the virtual augmented reality image of the medical device for output on the display.

In some embodiments, the one or more processors are located within the head-mounted device.

In some embodiments, the imaging means is a camera (e.g., infrared camera, RGB camera).

In some embodiments, the element is an identifying marker (e.g., reflective marker, optical code, machine-readable code) coupled to the medical device.

In accordance with several embodiments, an apparatus or system includes or consists essentially of a hands-free device (e.g., wearable device such as a head-mounted device) including a tracking means and a display and one or more processing devices. The tracking means is configured to track a physical location of a medical device and the display is configured to display a virtual augmented reality image of the medical device while a wearer can still see through the display. The one or more processors are configured to (e.g., upon execution of program instructions stored on one or more non-transitory computer-readable storage media): calculate a first location of the medical device at a first instance of time; calculate a second location of the medical device at a second instance of time; calculate respective further locations of the medical device at further instances of time; filter the further locations in response to a difference between the second location and the first location; and in response to the filtered further locations, generate the virtual augmented reality image of the medical device for output on the display.

In some embodiments, the tracking means is an image-capturing device (e.g., an infrared camera).

In some embodiments, the tracking means is an optical tracking device, an RFID reader, an NFC reader, a Bluetooth sensor, an electromagnetic tracking device, an ultrasound tracking device, or other tracking device.

Also described and contemplated herein is the use of any of the apparatus, systems, or methods for the treatment of a spine through a surgical intervention.

Also described and contemplated herein is the use of any of the apparatus, systems, or methods for the treatment of an orthopedic joint through a surgical intervention, including, optionally, a shoulder, a knee, an ankle, a hip, or other joint.

Also described and contemplated herein is the use of any of the apparatus, systems, or methods for the treatment of a cranium through a surgical intervention.

Also described and contemplated herein is the use of any of the apparatus, systems, or methods for the treatment of a jaw through a surgical intervention.

Also described and contemplated herein is the use of any of the apparatus, systems, or methods for diagnosis of a spinal abnormality or degeneration or deformity.

Also described and contemplated herein is the use of any of the apparatus, systems, or methods for diagnosis of a spinal injury.

Also described and contemplated herein is the use of any of the apparatus, systems, or methods for diagnosis of joint damage.

Also described and contemplated herein is the use of any of the apparatus, systems, or methods for diagnosis of an orthopedic injury.

In accordance with several embodiments, a method of presenting one or more images on a wearable display with reduced jitter is described and/or illustrated herein during medical procedures, such as orthopedic procedures, spinal surgical procedures, joint repair procedures, joint replacement procedures, facial bone repair or reconstruction procedures, ENT procedures, cranial procedures or neurosurgical procedures.

For purposes of summarizing the disclosure, certain aspects, advantages, and novel features of embodiments of the disclosure have been described herein. It is to be understood that not necessarily all such advantages may be achieved in accordance with any particular embodiment of the disclosure disclosed herein. Thus, the embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein. The methods summarized above and set forth in further detail below describe certain actions taken by a practitioner; however, it should be understood that they can also include the instruction of those actions by another party. The present disclosure will be more fully understood from the following detailed description of the examples thereof, taken together with the drawings.

### BRIEF DESCRIPTION OF THE DRAWINGS

Non-limiting features of some embodiments are set forth with particularity in the claims that follow. The following drawings are for illustrative purposes only and show non-limiting embodiments. Features from different figures may be combined in several embodiments. It should be understood that the figures are not necessarily drawn to scale. Distances, angles, etc. are merely illustrative and do not necessarily bear an exact relationship to actual dimensions and layout of the devices illustrated.

FIG. 1 is a schematic illustration of a medical procedure using augmented reality that is performed upon a patient, according to an embodiment of the disclosure,

FIG. 2 is a schematic illustration of an example head-mounted display (HMD) worn by a professional, according to an embodiment of the disclosure,

FIG. 3 is a schematic view of an image presented on a display, according to an embodiment of the disclosure,

FIG. 4 is a flowchart of steps of a jitter reduction process, according to an embodiment of the disclosure,

FIG. 5 is a flowchart of steps of a jitter-reduction algorithm, according to an embodiment of the disclosure.

FIG. 6 is a schematic figure illustrating an example HMD, according to another embodiment of the disclosure.

### DETAILED DESCRIPTION

### Overview

In an augmented reality medical procedure, a medical professional may wear a head-mounted display device (HMD) or other wearable or hands-free display device, such as a pair of spectacles, glasses, goggles, a single monocle or lens or a frame without lenses, other eyewear, or any other head-mounted device. The wearable or hands-free display device may be a non-head-mounted display/component or may include both head-mounted and non-head-mounted displays/components. The head-mounted device other wearable or hands-free display device may be a direct see-through device that comprises a display upon which are presented images (e.g., virtual icons, digital or virtual representations, graphics, images) of entities (e.g., surgical tools, surgical guides, diagnostic tools, introducer tools, implantation tools, implants, hardware, screws, rods, treatment instruments, cutting instruments, intact or removed anatomical portions of a patient, other objects) viewed by the professional while the professional can also see, through the display, at least a portion of the actual physical entities themselves. In accordance with several implementations it may be advantageous that, in the display, as viewed by the professional, the entities and their respective virtual images are aligned, e.g., by a predefined error, or within a predefined tolerance. In the following description, the entities are assumed to comprise a tool used by the professional, as well as a region of interest (ROI) of a patient undergoing the procedure. The systems and methods described herein may be used in connection with surgical procedures, such as spinal surgery, joint surgery (e.g., shoulder, knee, hip, ankle, other joints), orthopedic surgery, heart surgery, bariatric surgery, facial bone surgery, dental surgery, cranial surgery, or neurosurgery. The surgical procedures may be performed during open surgery or minimally-invasive surgery (e.g., surgery during which small incisions are made that are self-sealing or sealed with surgical adhesive or minor suturing or stitching). However, the systems and methods described may be used in connection with other medical procedures (including therapeutic and diagnostic procedures) and with other instruments and devices or other non-medical display environments. The devices and methods described herein are used to facilitate medical procedures, including both therapeutic and diagnostic procedures, and further include the performance of the medical procedures (including but not limited to performing a surgical intervention such as treating a spine, shoulder, hip, knee, ankle, other joint, jaw, cranium, etc.). Spinal procedures may be performed on any segment of the spine, including but not limited to, lumbosacral regions, sacral vertebrae, lumbar vertebrae, cervical vertebrae, iliosacral regions, thoracic vertebrae, etc.

In accordance with several embodiments, the tool is assumed to be used on the ROI, and in order that the tool image and the ROI image align (and optionally, also align respectively with their physical entities), the tool has a tool marker. The tool marker may be included in the tool or may be removably attached to the tool. There may also be a patient marker placed on or coupled to the patient, e.g., in the vicinity of the ROI. The patient marker may be configured to indicate, e.g., using a configured local vector, the ROI. In accordance with several embodiments, the tool marker and the patient marker are identifying markers for their respective associated entities.

According to some embodiments, the tool marker and the patient marker comprise one or more reflective elements which may be illuminated by a light source (e.g., an infra-red light source) which may be fixed to the HMD. At least one camera (e.g., infrared camera) may also be fixed to the HMD, and the camera may be configured to acquire images of the reflective elements of one or both of the two markers (the tool marker and the patient marker) or of at least a portion of the reflective elements of one or each of the two markers. One or more processors associated with the HMD may analyze the camera's acquired images so as to track the two markers (e.g., centroids of the markers), and thus the tool and the ROI, in a frame of reference of the HMD and/or camera. The processor(s) may then use the tracked locations of the tool and the ROI to implement the required alignment of a tool virtual image with an ROI virtual image and, optionally, with their physical entities (e.g., in augmented-reality or mixed reality systems).

During the procedure, there is typically motion of the tool and the ROI (typically to a lesser extent than the tool), and of their respective markers (e.g., tool marker and patient marker), correspondingly, with respect to the camera or other imaging or tracking device, and this motion is present in the images of the elements of the markers acquired by the camera or other imaging or tracking device. When HMDs having the camera mounted thereon are used, the motion may also derive from the camera itself (e.g., via the user's movements), while the captured and/or measured motion is the overall relative motion between the tool or ROI and the camera (e.g., the movement of the tool or ROI with respect to the camera). However, in some embodiments, because of the tracking implemented by the processor(s), the processor(s) is able to maintain the alignment described above, regardless of the above-described motion.

In practice, there is noise in the acquired reflective elements images, or images obtained from the reflective elements, leading to small variations in the analyzed position of the tool and of the ROI. These small variations may display as unwanted jitter in the virtual images of the tool and/or the ROI presented to the professional. For example, there may be a greater than desired misalignment for a period of time.

Embodiments of the disclosure address the problem of jitter by applying different low pass filters (e.g., filters that filter high-frequency signals, such as high frequency noise or jitter signals) to the two-dimensional (2D) positions or three-dimensional (3D) positions of the tool and ROI with respect to the camera (e.g., in the camera frame of reference) calculated based on the images captured by the camera. For example, when analysis of the reflective elements images indicates there is little or no motion of the markers (e.g., patient marker and/or tool marker), the processor(s) filters the position information (e.g., two dimensional (2D) positions or three dimensional (3D) positions of the tool and ROI) calculated based on the images of the camera via a low pass filter having a low cutoff frequency. According to some embodiments, the low cutoff frequency is between 0 Hz to 5 Hz (e.g., between 0 Hz to 3 Hz, between 1 Hz to 3 Hz, between 2 4 Hz, between 2 Hz and 5 Hz, overlapping ranges thereof or any value within the recited ranges, such as 1 Hz, 1.5 Hz, 2 Hz, 2.5 Hz, 3 Hz, 3.5 Hz, 4 Hz, 4.5 Hz, 5 Hz). Such a low pass filter inherently introduces latency or delay in the image presentation, but because there is little or no motion of the tool and the ROI with respect to the camera, such latency is acceptable in certain implementations.

However, when the reflective elements image analysis indicates there is motion of the markers (e.g., tool marker and/or patient marker), outside of (e.g., above or below) a predefined threshold, the latency referred to above may become unacceptable. In this case, e.g., during motion of the markers, the processor(s) may advantageously be programmed to filter the position information via a low pass filter having a high or higher cutoff frequency. According to some embodiments, the high cut off frequency is typically between 4 Hz to 10 Hz (e.g., 4 Hz to 7 Hz, 5 Hz to 8 Hz, 6 Hz to 10 Hz, overlapping ranges thereof, or any value within the recited ranges such as 5 Hz or 6 Hz). In some embodiments, the high cut off frequency is sufficiently higher than the low cut off frequency of the filter to filter out noise but not filter out desired signals.

In accordance with several embodiments, for the purpose of image presentation, a camera may acquire its images at a rate of 40 to 120 images per second. According to some embodiments, the camera frame rate is between 50 and 80 frames per second, such as 50, 60, 70 or 80 frames per second. The frame rate of the camera may be used to determine the frequency cutoff values. Embodiments of the invention advantageously build on this rate of image acquisition to ensure that when the reflective elements image analysis indicates a change from little or no motion to motion, the change of cutoff values in the low-pass filter is accomplished quickly, typically within one frame.

Several embodiments are particularly advantageous because they include one, several or all of the following benefits:
(i) reduction of jitter and/or noise in display of images of slow moving or stationary targets,
(ii) reduction of jitter and/or noise in images of fast moving targets,
(iii) maintenance of alignment between images of different entities regardless of jitter,
(iv) reduction of jitter and/or noise in images display without introducing undesired latency, and/or
(v) reduction of jitter and/or noise in images while keeping the display accurate, e.g., to allow navigation of displayed elements.

Although the present description specifically refers to an augmented-reality system, it may also apply, mutatis mutandis, to other image-guided systems employing virtual image presentation technology.

### SYSTEM DESCRIPTION

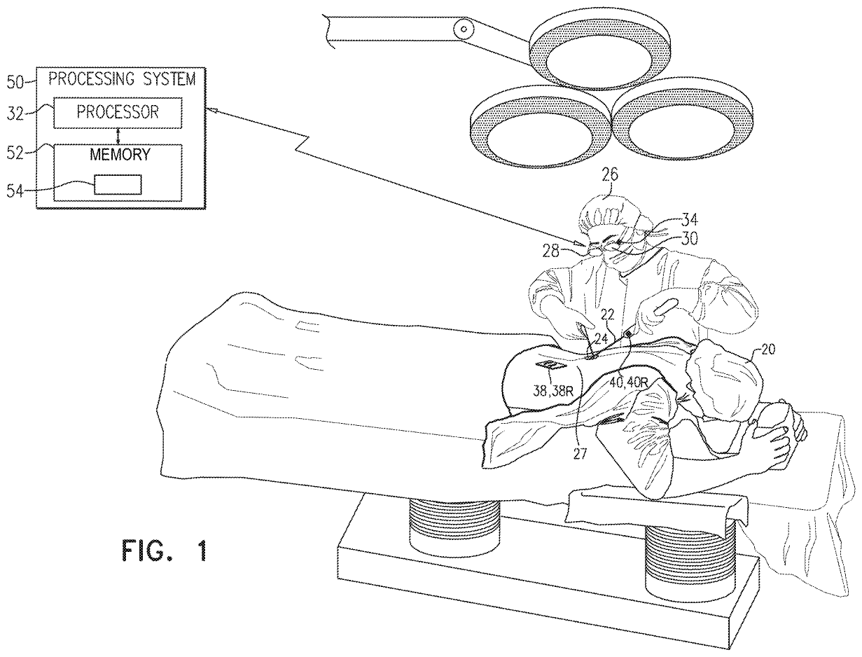

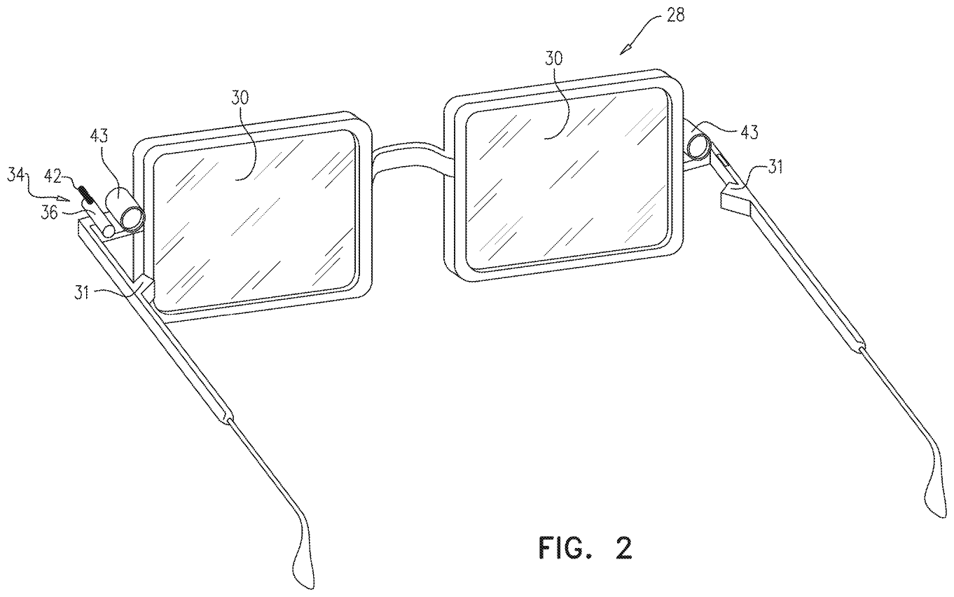

Reference is now made to FIG. 1, which is a schematic illustration of a medical procedure using augmented reality or mixed reality that is performed upon a subject 20, also herein termed patient 20, according to an embodiment of the disclosure. In the medical procedure shown in FIG. 1, a medical professional 26 uses a tool 22 to perform an action with respect to the patient's back 27, the tool being inserted via an incision 24 on the patient's back 27. Incision 24 is also assumed to be at least partially included in or adjacent to a region of interest (ROI), and is also referred to herein as ROI 24. Reference is also made to FIG. 2, which is a schematic illustration of a head-mounted device (HMD) 28 worn by professional 26, according to an embodiment.

Typically, HMD 28 includes one or more displays 30. The displays 30 enable professional 26 to observe a scene viewed through the displays 30 (as well as having images presented thereon), so that professional 26 acts as an observer. The displays 30 may include a combiner (e.g., optical combiner) that is controlled by a processing system 50 that includes a processor 32 interacting with a memory 52, and processing system 50 may be coupled wirelessly and/or via conductive or optical cables to HMD 28. Memory 52 comprises any computer-readable storage media configured to store computer-executable program instructions for a jitter-reduction algorithm 54, described in more detail below. Processor 32 may include one or more processing units. According to some embodiments, processor 32 or at least one processing unit of processor 32 may be installed in HMD 28. In some embodiments, at least one processing unit of processor 32 is not located on or in HMD 28. The display(s) 30 may facilitate a 3D stereoscopic view of augmented reality or mixed reality content while still allowing the professional 26 to see a live scene. The HMD 28 may provide elimination of attention shift and a reduced (e.g., minimized) line of sight interruption. In accordance with several embodiments, the HMD 28 may also be designed for close-up augmented reality vision where the virtual images are projected at 50 cm (30-60 cm or other distance to align with a typical focal distance of a person's eyes or a convergence when focusing) in order to reduce “vergence-accommodation conflict”, which can result in fatigue, headache, or general discomfort. For example, the light may appear to be coming from approximately 50 cm. The line of sight may be at approximately 30 degrees.

Typically, head-mounted device 28 includes a tracking device 34 that is configured to facilitate determination of the location and orientation of HMD 28 with respect to ROI 24 of the patient and/or with respect to tool 22. Tracking device 34 includes an image-capturing device 36 (e.g., a camera and herein referred to as camera 36), that is configured to image one or more reflective elements 38R of one or more patient markers 38 and/or one or more reflective elements 40R of one or more tool markers 40. According to some embodiments, capturing device 36 is a camera that is able to acquire images at the rate of 60 frames per second or more. As is described below, patient marker(s) 38 and tool marker(s) 40 act as identifying markers for respective entities associated with the markers.

Tracking device 34 may include a light source 42, which may be mounted on HMD 28. The light source 42 may be configured to irradiate the patient marker(s) 38 and the tool marker(s) 40, such that light reflects from marker reflective elements 38R and 40R toward camera 36. For some applications, image-capturing device 36 is a monochrome camera that includes a filter that is configured to only allow light to pass through that is of a similar wavelength to the light that is generated by the light source 42. For example, light source 42 may be an infrared light source (for example, a light source that generates light at a wavelength of between 700 nm and 800 nm) and the image-capturing device 36 may include a corresponding infrared filter. Optionally, the HMD 28 may include additional image-capturing devices (e.g., cameras 43), such as shown in FIG. 2, which are configured to capture images of scenes in the visible spectrum (e.g., red-green-blue (RGB) cameras).

In some embodiments, the tracking device 34 may include optical imaging devices, ultrasound imaging devices, thermal imaging devices, radar imaging devices, or other imaging devices configured to capture still images and/or video images or one or more reading devices. In some embodiments, the tracking device 34 may additionally or alternatively include one or more non-imaging devices for tracking locations of devices, such as radiofrequency identification (RFID) readers, near field communication (NFC) readers, optical code readers. detectors or scanners, Bluetooth sensors, position sensors, electromagnetic sensors, or other wireless sensing devices.

As stated above, tool marker(s) 40 comprises tool marker reflective elements 40R, and images of these markers 40 acquired by camera 36 enable processor 32 to track the location and orientation of tool(s) 22 with respect to camera 36. In addition, patient marker 38 comprises patient marker reflective elements 38R, and the patient marker 38 is configured to indicate, e.g., by an associated local vector, the position of ROI 24, so that processor 32 is able to use the images of elements 38R to track the position of ROI 24 with respect to camera 36. Patient marker(s) 38 and/or tool marker(s) 40 may be (e.g., may incorporate structural and operational features) according to the markers described in U.S. Pat. No. 10,939,977 to Messinger et al. and 10,835,296 to Elimelech et al., and in US Patent Publication No. 2023/0009793 to Gera et al., the content of each of which is incorporated herein by reference. Although patient marker 38 shown in FIG. 1 is in the form of a marker directly attached to a skin of patient 20, other marker types which may be attached to patient 20 may be utilized, e.g., via a mounting device such as a clamp or a pin mounted to a bone of the patient, and as described, for example, in U.S. Pat. Nos. 10,939,977, 10,835,296 and US Patent Publication No. 2023/0009793 incorporated hereinabove.

In accordance with several embodiments, the image presented on display 30 includes a computer-generated image (e.g., a virtual image) that is projected onto the display 30 by a projector 31 (e.g., one or more projectors). The projected image may include images of tool 22 and/or ROI 24 that have been stored in memory 52 and that are retrieved by processor 32. The images may be provided in a 3D stereoscopic view. The virtual images may be projected at 50 cm (or other distance designed to align with a normal focal distance of a person's eyes) in order to reduce “vergence-accommodation conflict”, which can result in fatigue, headache, or general discomfort. The line of sight may be at approximately 30 degrees. By virtue of the tracking of tool 22 and ROI 24 described above, processor 32 is able to align the stored image of tool 22 with the stored image of ROI 24 and incorporate the combined images into the computer-generated image projected on display 30. Furthermore, processor 32 is able to overlay and align the stored images of tool 22 and ROI 24 with the actual tool 22 and ROI 24 as viewed by user 26 through display 30. The display 30 may be configured to provide the display directly onto a retina of the wearer of the HMD 28. The

### EXAMPLE DISPLAY IMAGE AND JITTER REDUCTION TECHNIQUES

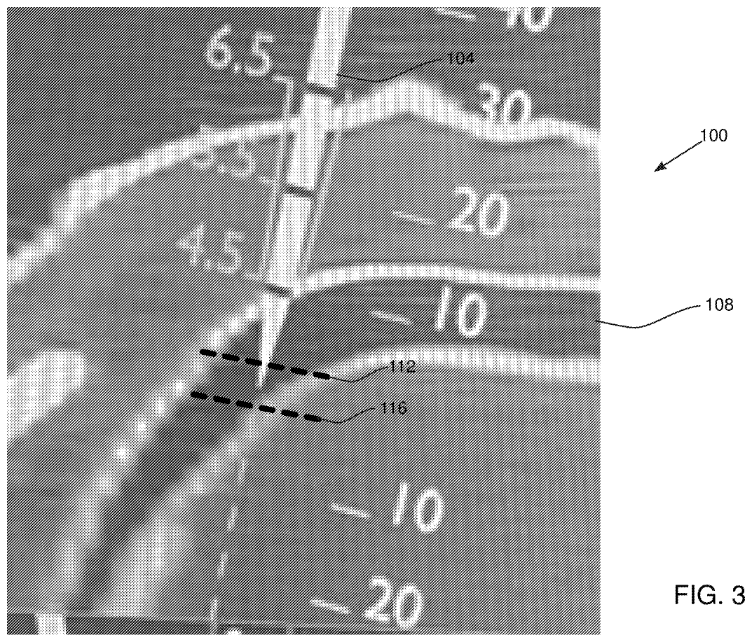

FIG. 3 is a schematic view of an image 100 presented on display 30, according to one embodiment. Image 100 comprises a virtual image 104 of tool 22 that processor 32 has retrieved from memory 50, and that the processor 32 has positioned within image 100 according to the position of the tool 22 ascertained from the acquired images of tool marker elements 40R. Image 100 also includes an image 108 of ROI 24.

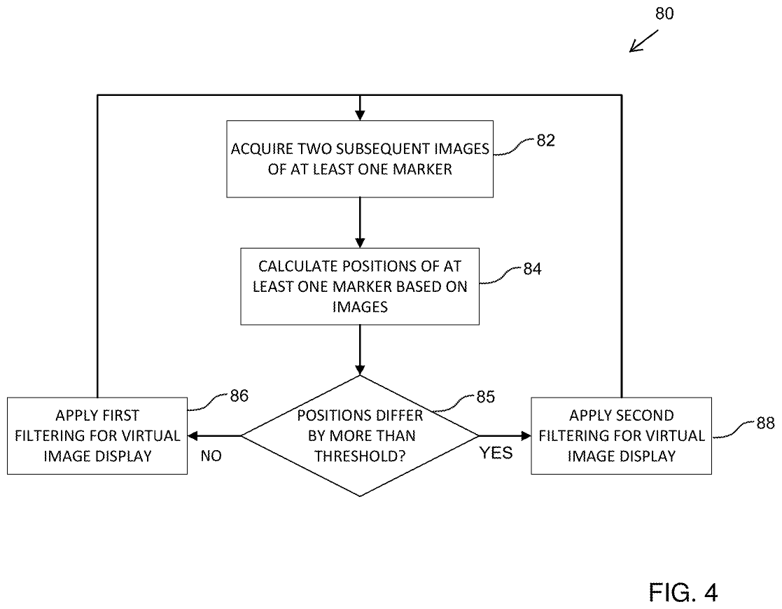

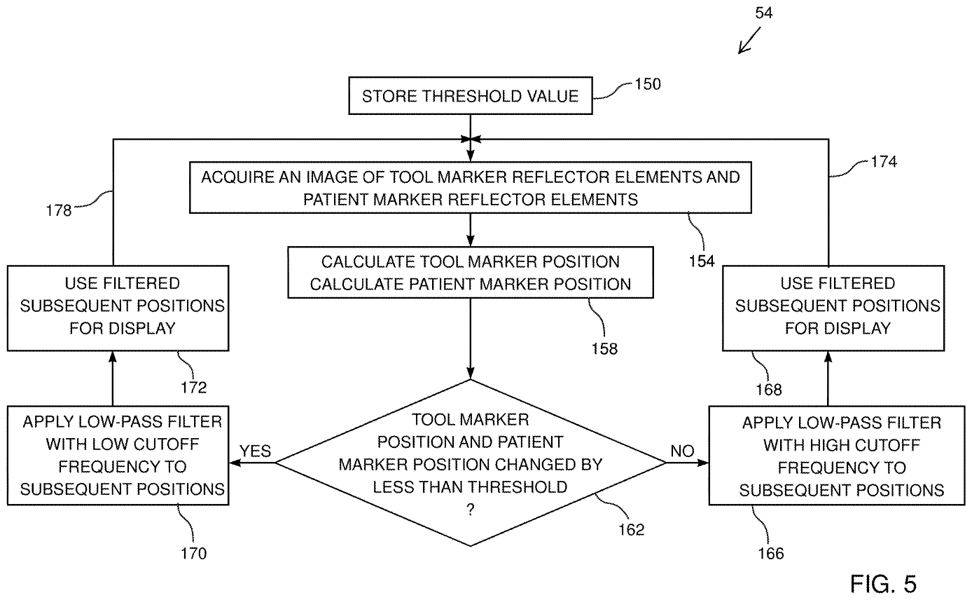

Because of the noise associated with the acquisition of tool marker elements 40R and patient marker elements 38R, the inventors have found that the calculated position of tool 22 with respect to ROI 24 may also be subject to noise, and this noise is apparent in jitter of image 104 with respect to image 108. Thus, the tip of image 104, for example, may oscillate between positions identified by dashed lines 112 and 116 in image 108. To overcome the jitter, during the medical procedure referred to above, processor 32 is configured or programmed to execute one or more jitter-reduction algorithms, processes or techniques (e.g., jitter-reduction algorithm 54) stored in memory communicatively coupled to the processor 32. Steps of process 80 and algorithm 54 are described below with reference to the flowcharts of FIGS. 4 and 5.

In accordance with several embodiments, use of jitter reduction techniques (e.g., low pass filters with varying cutoff frequencies) results in a desired smooth signal that reduces jitter but also results in increased latency, which may not be as desirable. Accordingly, in accordance with several embodiments, the jitter reduction techniques (e.g., one or more low pass filters with varying cutoff frequencies) may be controlled so that the jitter reduction techniques (e.g., one or more low pass filters with higher cutoff frequencies) that result in increased latency are not running continuously all the time but are instead applied (e.g., activated) at selected periods of time (e.g., time periods when the images being detected are changing or are dynamic enough to warrant more stringent jitter reduction methods being used). The less stringent jitter reduction (e.g., low pass filtering with a first, lower cutoff frequency) may be used (e.g., activated or applied) when a scene is determined to be static (as determined by evaluation of one or more thresholds) and the more stringent jitter reduction (e.g., low pass filtering with a second, higher cutoff frequency) may be used (e.g., may be deactivated) when a scene is determined to be dynamic (as determined by evaluation of the one or more thresholds). In accordance with several embodiments, the latency is advantageously not detectable by an operator or at least is not significantly or noticeably increased as a result of the controlled jitter reduction techniques, which may be fully or partially automated by one or more processors. The cutoff frequencies may be prestored or predetermined or may be input by a user or automatically adjusted over time based on the images (e.g., by computer processing techniques and algorithms, such as trained algorithms or trained neural networks).

In several embodiments, a method for reducing jitter includes determining whether the position or location of one or more entities being tracked no longer satisfies one or more threshold criteria (e.g., the position or location has changed by more than a threshold amount or value as determined from one or more images). If it is determined that the position or location does not satisfy or meet one or more threshold criteria, then a first level of (e.g., increased or more strict) jitter reduction may be applied. If it is determined that the position or location satisfies or meets one or more threshold criteria, then a second level of (e.g., reduced or less strict) jitter reduction may be applied. The new positions may be stored and the method may be repeated iteratively.

In some embodiments, the jitter reduction techniques or methods involve application of one or more low pass filters (e.g., IIR filters or other digital or other noise filters). The increased or more strict jitter reduction may involve one or more low pass filters with a first (e.g., higher) cutoff frequency than a second (e.g., lower) cutoff frequency of the reduced or less strict jitter reduction. The cutoff frequencies may be preset or predetermined or may be adjusted either manually or automatically in real time over time (e.g., by computer processing techniques and algorithms, such as trained algorithms or trained neural networks).

In some embodiments, the filtering with the lower cutoff frequency during the second level, reduced or less strict, jitter reduction also results in reduced latency. Thus, applying the different levels of jitter reduction based on threshold criteria may advantageously increase user experience and improve performance and outcome.

In accordance with several embodiments, a method for reducing jitter in display presentations during image-guided surgery (e.g., spinal surgery or other surgical procedures) or image-guided medical procedures (including non-surgical therapeutic procedures or in diagnostic procedures) may include acquiring a first image of reflective elements of a first identifying marker coupled to an instrument or tool configured for use in a spinal surgical procedure or other medical procedure. For example, a processor may cause a camera (e.g., infrared camera) to acquire the first image. The camera may be on a wearable tracking and display device worn by a professional performing the surgical or other medical procedure. The method may also include calculating a first position of the first identifying marker in response to the first image. The method may also include acquiring, subsequent to acquiring the first image, a second image of the reflective elements of the first identifying marker and calculating a second position of the first identifying marker in response to the second image. The method may also include acquiring, subsequent to acquiring the second image, further images of the reflective elements of the first identifying marker and calculating respective further positions of the first identifying marker in response to the further images. The method may further include filtering the further positions in response to a difference between the second position and the first position and, in response to the filtered further positions, generating images of at least a portion of the instrument or tool for output on the wearable tracking and display device to facilitate performance of the image-guided surgical or other medical procedure by the professional.

The calculations of the various positions of the first identifying marker (e.g., a tool marker) may also involve determination of locations of a second identifying marker (e.g., patient marker) coupled to a patient and positioned in proximity to a region of interest of the patient associated with the spinal surgical procedure. The second identifying marker may also include reflective elements that can be included in the acquired images. The positions of the first identifying marker may be calculated with respect to, or using as a reference, the locations of the second identifying marker. The second identifying marker may be a marker having a local vector indicative of the region of interest.

In some embodiments, when the difference between the second position and the first position is not greater than a threshold value, the filtering comprises applying low-pass filtering with a first cutoff frequency, and when the difference is greater than the threshold value, the filtering comprises applying low-pass filtering with a second cutoff frequency greater than the first cutoff frequency.

In some embodiments, the first and the second positions are two-dimensional positions measured in pixels, and the difference between the second position and the first position is a velocity from the first position to the second position measured in pixels per second or in mm/sec. In some embodiments, the first and the second positions are three-dimensional positions, and the difference between the second position and the first position is a velocity from the first position to the second position.

The threshold value may be a stored preset value that is provided by a user or that is automatically calculated by one or more processors (e.g., by computer processing techniques and algorithms, such as trained algorithms or trained neural networks). The threshold value may remain constant or may change.

In various embodiments, the second higher cutoff frequency is a value between 4 Hz and 7 Hz and the first lower cutoff frequency comprises a value between 0 Hz and 3 Hz. The cutoff frequencies may be preset and constant or may be input by a user or automatically adjusted over time (e.g., by computer processing techniques and algorithms, such as trained algorithms or trained neural networks).

FIG. 4 is a flowchart of an example implementation of a jitter reduction process 80. The jitter reduction process 80 may be executed by one or more processors (e.g., one or more processors of a wearable, hands-free, and/or head-mounted tracking and display device), a non-head-mounted display/component or both head-mounted and non-head-mounted displays/components. The process 80 begins at block 82 by acquiring two subsequent images of at least one marker. For example, the one or more processors may cause a camera (e.g., infrared camera) to acquire images of the at least one marker. The images may be based on reflective elements of the at least one marker. The at least one marker may be a tool marker configured to be attached to a tool for use in a medical procedure (e.g., surgical procedure, such as a spinal surgical procedure, a non-surgical procedure, and/or a diagnostic procedure). The at least one marker may also include a patient marker configured to be attached to a portion of the patient associated with the medical procedure.

The process 80 continues at block 84 by calculating positions of the at least one marker based on the acquired images at block 82. The positions may be calculated based on a registration step previously performed in connection with the at least one marker. The positions may be calculated based on 2D positions or on 3D positions.

At decision block 85, the process 80 involves determining (e.g., calculating) whether the calculated positions differ by more than a threshold value. If 2D positions are used, the threshold value may be defined in terms of velocity and as a number of pixels per second (e.g., measured based on the images). If 3D positions are used, the threshold value may be defined in terms of velocity and as a number of distance (e.g., millimeters) per second (e.g., measured via the camera). The threshold value may be preset and stored in memory (e.g., memory 52). The threshold value may be input by a user or may be prestored at the time of manufacture or assembly. The threshold value may remain constant or may be changed. If the calculated positions do not differ by more than the threshold value, then, proceeding to block 86, a first filtering is applied for generating the virtual image output for presentation on the display. The process 80 may then repeat as further images are acquired. The process 80 may be repeated immediately or after a defined period of time. If the calculated positions do differ by more than the threshold value at decision block 85, then, proceeding to block 88, a second filtering is applied for generating the virtual image output for presentation on the display. The process 80 may be repeated immediately or after a defined period of time.

As discussed above, the first filtering may involve low-pass filtering with a first cutoff frequency and the second filtering may involve low-pass filtering with a second cutoff frequency higher than the first cutoff frequency. The second filtering may result in increased latency. Accordingly, by repeating the process 80, the first filtering may be applied during time periods when the markers are not appreciably changing positions (e.g., the scene being imaged or viewed is relatively static) so as to reduce latency when the second filtering may not be needed, thereby improving user experience by removing high frequency noise.

FIG. 5 is a flowchart of steps of one example implementation of jitter-reduction algorithm 54. In one embodiment, algorithm 54 is stored in memory 52 as a state machine, but any convenient method for storing steps of the algorithm 54 may be used. Processor 32 (which again may comprise one or more processors or processing units) activates the algorithm 54 during a medical procedure, such as referred to above, and it will be appreciated that the algorithm 54 comprises an iterative process. In several embodiments, the algorithm or process 54 is applied to each marker in a separate manner or may be applied to one or more markers (e.g., a first identifying marker and second identifying marker). The process is herein assumed to iterate as each frame of a scene is acquired by camera 36, as will be described hereinbelow.

In an initial step 150, one or more threshold criteria (e.g., a threshold value or condition) to be used in the algorithm 54 is stored in memory 52. The threshold value or condition may be a single value or condition or may include multiple threshold criteria. The threshold criteria may not be a value but may include one or more Boolean conditions or other criteria. The threshold criteria may include both one or more values and one or more conditions. The threshold value may be input by an operator, may be a predefined standard value in memory 52, or may be automatically determined by the processor 32 (and may be adjusted in real time over time (e.g., by computer processing techniques and algorithms, such as trained algorithms or trained neural networks)).

According to some embodiments, the filtering is performed on 2D positions and the threshold value is defined in terms of velocity and as a number of pixels per second (e.g., measured based on the image). According to some embodiments, the filtering is performed on 3D positions, and the threshold value is defined in terms of velocity and as a number of distance (e.g., millimeters) per second (e.g., measured via tracking device 34). Processor 32 uses the threshold value to check the level of movement in the image or frame of each marker, or one or more markers but not all markers. In some embodiments, the threshold value is set between two to ten pixels per second (e.g., two to eight pixels per second, two to six pixels per second, three to nine pixels per second, four to ten pixels per second, four to eight pixels per second, four to six pixels per second, overlapping ranges thereof, or any value within the recited ranges). For example, the threshold value may be set to four, five or six pixels per second. In some embodiments, the threshold value is set between five and twenty mm/sec (e.g., between five and fifteen mm/sec, between five and ten mm/sec, between ten and twenty mm/sec, overlapping ranges thereof, or any value within the recited ranges). Other threshold values may be used as desired and/or required.

When the threshold value is set in pixels per second, the effect of applying the value may depend on the distance between the imaging device and the entities being imaged or otherwise tracked. When the threshold value is set as a distance (e.g. mm) per second, the effect of applying the threshold is not dependent on the distance between the imaging device and the entities being imaged or otherwise tracked.

In an element acquisition step 154 and a position step 158, the one or more entities are tracked by the tracking device 34 and positions of the one or more entities are calculated or otherwise determined, respectively, and the positions are stored in memory (e.g., memory 54).

In some embodiments involving imaging devices and markers with reflector elements as reflected in FIG. 5, a camera or other image-capturing device 36, in acquiring a frame of a scene viewed by the camera or other image-capturing device, acquires images comprising tool marker elements 40R and patient marker elements 38R. At step 158, processor 32 uses the frame to calculate the positions of each of tool marker elements 40R and patient marker elements 38R, and, from those positions, the processor 32 calculates a position for the tool marker 40 and for the patient marker 38 with respect to camera 36. The processor 32 stores the position of the tool marker 40 and the patient marker 38. In some implementations, positions for each of the marker elements 40R and/or 38R are not required to be calculated but instead one or more, but less than all, positions are calculated or otherwise determined.

In a decision step 162, the processor 32 checks to determine if the calculated positions of the one or more entities have changed by an amount that does not satisfy the one or more threshold criteria (e.g., exceeds a stored threshold value or does not satisfy a threshold condition).

For example, with reference to the specific implementation of FIG. 5, the processor 32 checks the calculated positions for each one of the tool marker 40 and the patient marker 38, separately, and determines whether both the position (e.g., via velocity) of the marker 38 and 40, as calculated and stored in step 158, has changed, by less than (or less than or equal to) the threshold value stored in the initial step. The change may be calculated by comparing the present position with the respective one or more positions stored with respect to one or more previous frames acquired by camera or other image-capturing device 36.

If the outcome of step 162 does not fall outside the threshold condition (e.g., returns a negative value), such as the calculated position of the marker 38 and/or 40 has changed by more than the threshold value, control continues to a filtering step to apply jitter reduction filtering with a first type of filtering. For example, as shown in FIG. 5, the process 54 proceed to a low pass filter step 166, wherein the processor 32 applies a low pass filter with a high cutoff frequency (e.g., a first frequency that is higher than a second, lower cutoff frequency but may still be a relatively low cutoff frequency). The filter with the high cutoff frequency may then be applied to subsequent positions of the one or more entities (e.g., markers 38, 40, which may be determined in in subsequent frames).

From step 166, control continues at a display step 168, wherein the processor 32 uses the high cutoff filtered marker positions when presenting images of the tool 22 and/or the ROI 24 on display 30.

If at step 162, the processor 32 determines that the change in position does not fall outside the threshold condition (e.g., returns a positive value), such as the calculated position of the tool marker and the patient marker has not changed by more than the threshold value, control continues to a low pass filter step 170, wherein the processor 32 applies a low pass filter with a low cutoff frequency (e.g., a second frequency that is lower than the high cutoff frequency). In this path, the filter with the low cutoff frequency is also applied to subsequent positions of the one or more entities (e.g., markers 38, 40), which may be determined in subsequent frames.

According to some embodiments, the low pass filter utilizing a low cutoff frequency and/or the low pass filter utilizing a high cutoff frequency is an infinite impulse response (IIR) filter (e.g., IIR first order filter). However, other appropriate types of filters (e.g., digital filters) may be used.

According to some embodiments, a low pass-filter with low cutoff frequency may be applied if at least the position of one of the tool marker or patient marker is changed by less than the threshold.

From step 170 control continues at a display step 172, wherein the processor 32 uses the low cutoff filtered marker positions when presenting images of the tool 22 and/or the ROI 24 on display 30.

From steps 168 and 172 control returns to element acquisition step 154, as indicated by arrows 174, 178, and the processor 32 reiterates the steps of the flowchart (algorithm 54).

In the reiteration path indicated by arrow 178 that includes steps 170 and 172, the low cutoff frequency may introduce latency into the positions calculated by processor 32, but this latency may be considered acceptable since there is little or no movement of the positions.

According to some embodiments, a bias is introduced by decision step 162 towards movement of higher velocity. For example, during reiterations, the processor 32 may average the marker velocities (e.g., from a preceding frame or image to a current frame or image) calculated during a predetermined time window or for each predetermined number of frames or images. In one example, the averaging may comprise applying a moving average of approximately 60 frames. In accordance with several implementations, only if the averaged velocity is equal or below the threshold, a low-pass filter having a low cutoff frequency will be applied (step 170).

In one example, the low cutoff frequency is between 0 and 3 Hz (e.g., 0 Hz, 0.5 Hz, 1 Hz, 1.5 Hz, 2 Hz, 2.5 Hz, 3 Hz). In other examples, the low cutoff frequency may be higher than 3 Hz but lower than the high cutoff frequency.

However, in some implementations, it is only required for one frame to display a velocity above the threshold to apply the low pass filter with a high cutoff frequency (step 166). The high cutoff frequency introduces little latency into the positions calculated by processor 32, and while the jitter is not reduced by as much as by the low cutoff frequency, this jitter may or may not be acceptable since there is movement.

In one example, the high cutoff frequency is between 4 and 10 Hz (e.g., 4 Hz, 4.5 Hz, 5 Hz, 5.5 Hz, 6 Hz, 6.5 Hz, 7 Hz, 7.5 Hz, 8 Hz, 8.5 Hz, 9 Hz, 9.5 HZ, 10 Hz). In other examples, the high cutoff frequency is lower than 4 Hz or higher than 4 Hz but higher than the first cutoff frequency.

The algorithm or method described in FIG. 5 refers by way of example to an image of a tool marker and patient marker reflector elements and may be applied, mutatis mutandis, to an image of one or more other tracked entities, such as those described herein.

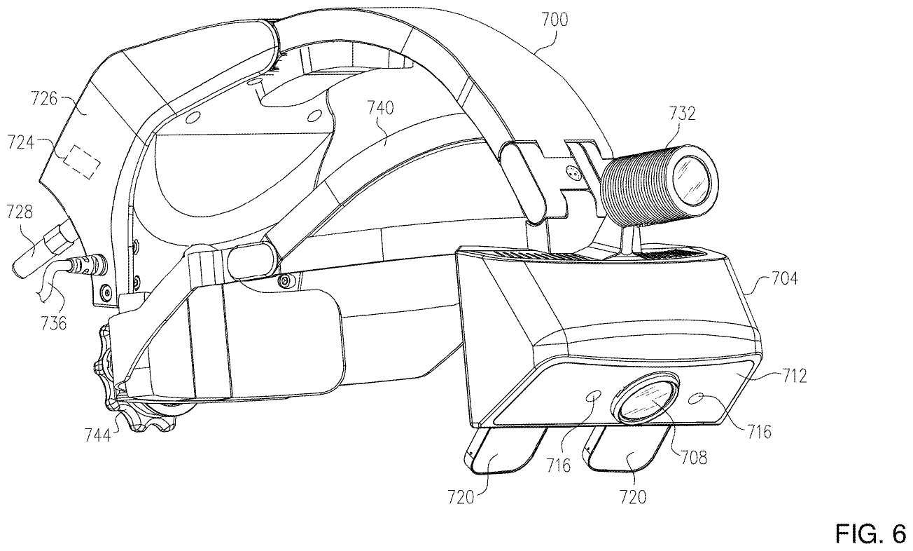

FIG. 6 is a schematic figure illustrating an example head-mounted device (HMD) 700, according to an embodiment of the disclosure. HMD 700 is worn by professional 26, and may be used in place of device 28 (FIG. 2). HMD 700 comprises an optics housing 704 which incorporates an infrared camera 708. Housing 704 also comprises an infrared transparent window 712, and within the housing, e.g., behind the window, are mounted one or more infrared projectors 716. Mounted on housing 704 are a pair of augmented reality displays 720, which allow professional 26 to view entities, such as part or all of ROI 24 through the displays 720, and which are also configured to present to the professional images (e.g., virtual images, virtually augmented images, augmented reality images and/or mixed reality images) that may be received from processing system 50 or any other information. The display(s) 30 may facilitate a 3D stereoscopic view of augmented reality or mixed reality content while still allowing the professional 26 to see a live scene. The HMD 700 may provide elimination of attention shift and a reduced (e.g., minimized) line of sight interruption. In accordance with several embodiments, the HMD 700 may also be designed for close-up augmented reality vision where the virtual images are projected at 50 cm in order to reduce “vergence-accommodation conflict”, which can result in fatigue, headache, or general discomfort. The line of sight may be at approximately 30 degrees.

The HMD 700 includes a processor 724, mounted in a processor housing 726, which operates elements of the HMD 700. Processor 724 may communicate with processing system 50 via an antenna 728, although in some embodiments processor 724 may perform some of the functions performed by processing system 50, and in other embodiments may completely replace processing system 50. Processor 724 may include one or more processors or processing units.

Mounted on the front of HMD 700 is a flashlight 732. The flashlight projects visible spectrum light onto objects so that professional 26 is able to clearly see the objects through displays 720. Elements of the head-mounted device 700 are typically powered by a battery (not shown in the figure) which supplies power to the elements via a battery cable input 736.

HMD 700 is held in place on the head of professional 26 by a head strap 740, and the professional 26 may adjust the head strap 740 by an adjustment knob 744.

The embodiments described above may be applied with one or more identifying markers for one or more respective associated entities. The embodiments described above may be applied with different filters for different markers and/or with different cutoff values for different markers. The embodiments described above may be applied to different medical procedures utilizing virtual image presentation and are not limited to spinal procedures. The embodiments described above may also be applied to visualization techniques not associated with medical procedures.

In some implementations, the system comprises various features that are present as single features (as opposed to multiple features). For example, in one embodiment, the system includes a single HMD, a single camera, a single tool, a single patient marker, a single tool marker, a single display. Multiple features or components are provided in alternate embodiments.

In some implementations, the system comprises one or more of the following: means for imaging (e.g., one or more cameras, MRI machines, CT imaging machine, fluoroscope), means for introduction or performing surgery (e.g., introducer cannula, screwdriver, surgical tool, insertion tool, saw, bone cutting tool, ablation device), means for tracking (e.g., one or more cameras and light sources), means for processing (e.g., one or more specific-purpose processors), means for storing (e.g., random access or read-only memory), means for registration (e.g., adapters, patient markers, tool markers), means for tracking (e.g., one or more imaging devices, one or more electromagnetic tracking device, one or more RFID tracking devices, one or more NFC tracking devices, one or more optical tracking devices, one or more LIDAR tracking devices, etc.).

Any of the method steps described herein may be performed by one or more hardware processors by executing program instructions stored on a non-transitory computer-readable medium. The systems can include multiple engines or modules for performing the processes and functions described herein, such as the process 80 and the algorithm 54 described above. The engines or modules can include programmed instructions for performing processes as discussed herein. The programming instructions can be stored in a memory. The programming instructions can be implemented in C, C++, Python, JAVA, or any other suitable programming languages. In some embodiments, some or all of the portions of the systems including the engines or modules can be implemented in application specific circuitry such as ASICs and FPGAs. The processors described herein may include one or more central processing units (CPUs) or processors or microprocessors. The processors may be communicatively coupled to one or more memory units, such as random-access memory (RAM) for temporary storage of information, one or more read only memory (ROM) for permanent storage of information, and one or more mass storage devices, such as a hard drive, diskette, solid state drive, or optical media storage device. The processors (or memory units communicatively coupled thereto) may include modules comprising program instructions or algorithm steps configured for execution by the processors to perform any of all of the processes or algorithms discussed herein. The processors may be communicatively coupled to external devices (e.g., display devices, data storage devices, databases, servers, etc. over a network via a network communications interface. In general, the algorithms or processes described herein can be implemented by logic embodied in hardware or firmware, or by a collection of software instructions, possibly having entry and exit points, written in a programming language, such as, for example, Python, Java, Lua, C, C#, or C++. A software module or product may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the computing system 50, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware. Generally, any modules or programs or flowcharts described herein may refer to logical modules that may be combined with other modules or divided into sub-modules despite their physical organization or storage.

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section. The various features and processes described above may be used independently of one another or may be combined in various ways. All possible combinations and sub-combinations are intended to fall within the scope of this disclosure. No single feature or group of features is necessary or indispensable to each and every embodiment. In addition, certain method or process blocks or steps may be omitted in some implementations. The methods and processes described herein are also not limited to any particular sequence, and the blocks, steps, or states relating thereto can be performed in other sequences that are appropriate. For example, described blocks, steps, or states may be performed in an order other than that specifically disclosed, or multiple blocks or states may be combined in a single block or state. The example blocks, steps, or states may be performed in serial, in parallel, or in some other manner. Blocks, steps, or states may be added to or removed from the disclosed example embodiments. Any process descriptions, elements, or blocks in the flow diagrams described herein and/or depicted in the attached figures should be understood as potentially representing modules, segments, or portions of code which include one or more executable instructions for implementing specific logical functions or steps in the process.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication.

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 0 to 5 Hz should be considered to have specifically disclosed subranges such as from 0 to 2 Hz, from 1 to 3 Hz, from 2 to 4 Hz, from 3 to 5 Hz, etc., as well as individual numbers within that range, for example, 0, 1, 1.5, 2, 2.5, 3, 3.5, 4, 4.5, 5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 2:1” includes “2:1.” As used herein, the terms “about” or “approximately” for any numerical values or ranges of an entity or an element indicate a suitable dimensional tolerance that allows the entity or element to function for its intended purpose as described herein. More specifically, “about” or “approximately” may refer to the range of values +20% of the recited value, e.g. “about 90%” may refer to the range of values from 72% to 108%, and “approximately 60 frames” may refer to the range of values from 48 frames to 72 frames.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

1. A system for reducing jitter in display presentations during image-guided medical procedures, the system comprising:
an identifying marker having reflective elements thereon configured to be coupled to an instrument or tool configured for use in a medical procedure;
a wearable device comprising a camera configured to acquire images of the reflective elements of the identifying marker and a display configured to display a virtual augmented reality image of at least a portion of the instrument or tool while a wearer of the wearable device can still see through the display; and
one or more processors configured to:
operate the camera to acquire a first image of the reflective elements and to calculate a first position of the identifying marker in response to the first image;
operate the camera, subsequent to acquiring the first image, to acquire a second image of the reflective elements and to calculate a second position of the identifying marker in response to the second image;
operate the camera, subsequent to acquiring the second image, to acquire further images of the reflective elements and to calculate respective further positions of the identifying marker in response to the further images;
filter the further positions in response to a difference between the second position and the first position; and
in response to the filtered further positions, generate the virtual augmented reality image of at least the portion of the instrument or tool on the display of the wearable device.

2. (canceled)

3. (canceled)

4. (canceled)

5. The system of claim 1, wherein:
the wearable device comprises a head-mounted device;
the camera comprises an infrared camera;
wherein when the difference is not greater than a preset threshold, the filtering comprises applying a first low-pass filter with a first preset cutoff frequency, and when the difference is greater than the preset threshold, the filtering comprises applying a second low-pass filter with a second preset cutoff frequency greater than the first cutoff frequency;
the second cutoff frequency comprises a value between 4 Hz and 7 Hz; and
the first cutoff frequency comprises a value between 0 Hz and 3 Hz.

6. The system of claim 1, wherein the first and the second positions are two-dimensional positions measured in pixels, and wherein the difference between the second position and the first position comprises a velocity from the first position to the second position measured in pixels per second.

7. The system of claim 1, wherein the first and the second positions are three-dimensional positions, and wherein the difference between the second position and the first position comprises a velocity from the first position to the second position.

8. (canceled)

9. (canceled)

10. The system of claim 1, wherein when the difference is not greater than a preset threshold, the filtering comprises applying a first low-pass filter with a first preset cutoff frequency, and when the difference is greater than the preset threshold, the filtering comprises applying a second low-pass filter with a second preset cutoff frequency greater than the first preset cutoff frequency, and wherein the second preset cutoff frequency comprises a value between 3 Hz and 10 Hz.

11. The system of claim 1, wherein when the difference is not greater than a preset threshold, the filtering comprises applying a first low-pass filter with a first preset cutoff frequency, and when the difference is greater than the preset threshold, the filtering comprises applying a second low-pass filter with a second preset cutoff frequency greater than the first preset cutoff frequency, and wherein the second preset cutoff frequency comprises a value between 4 Hz and 7 Hz.

12. (canceled)

13. The system of claim 1, wherein when the difference is not greater than a preset threshold, the filtering comprises applying a first low-pass filter with a first preset cutoff frequency, and when the difference is greater than the preset threshold, the filtering comprises applying a second low-pass filter with a second preset cutoff frequency greater than the first preset cutoff frequency, and wherein the first low-pass filter and the second low-pass filter are infinite impulse response filters.

14. (canceled)

15. (canceled)

16. (canceled)

17. (canceled)

18. The system of claim 1, wherein, when the difference is not greater than a threshold value, the filtering comprises applying filtering with a first cutoff frequency, and when the difference is greater than the threshold value, the filtering comprises applying filtering with a second cutoff frequency greater than the first cutoff frequency, and wherein the second cutoff frequency comprises a value between 4 Hz and 7 Hz, and wherein the first cutoff frequency comprises a value between 0 Hz and 3 Hz.

19. (canceled)

20. (canceled)

21. (canceled)

22. The system of claim 1, wherein, when the difference is not greater than a threshold value, the filtering comprises applying filtering with a first cutoff frequency, and when the difference is greater than the threshold value, the filtering comprises applying filtering with a second cutoff frequency greater than the first cutoff frequency, and 14, wherein the filtering comprises use of one or more low-pass filters.

23. The system of claim 22, wherein the one or more low-pass filters are one or more infinite impulse response filters.

24. The system of claim 23, wherein the one or more infinite impulse response filters are first order filters.

25. The system of claim 1, further comprising a second identifying marker, wherein the second identifying marker has a local vector indicative of a region of interest associated with the medical procedure.

26. The system of claim 25, wherein the second identifying marker is configured to be coupled to a portion of a patient at or near the region of interest.

27. The system of claim 26, wherein the second identifying marker comprises reflective elements configured to be imaged by the camera so as to facilitate determination of a location of the second identifying marker that is used by the one or more processors to calculate the first position, the second position, and the further positions.

28. A system for reducing jitter in display presentations during image-guided surgery, the system comprising:
an first identifying marker having reflective elements thereon configured to be coupled to an instrument or tool configured for use in a spinal surgical procedure;
a second identifying marker having reflective elements thereon configured to be coupled to a patient at or adjacent a region of interest associated with the spinal surgical procedure;
a head-mounted device comprising an infrared camera configured to acquire images of the reflective elements of the first identifying marker and the second identifying marker and a see-through display configured to display virtual augmented reality images of at least a portion of the instrument or tool while a wearer of the head-mounted device can still see through the display; and
one or more processors configured to, upon execution of instructions stored on one or more non-transitory computer readable media:
operate the infrared camera to acquire a first image of the reflective elements of the first identifying marker and the second identifying marker and to calculate a first position of the first identifying marker in response to the first image;
operate the camera, subsequent to acquiring the first image, to acquire a second image of the reflective elements reflective elements of the first identifying marker and the second identifying marker and to calculate a second position of the first identifying marker in response to the second image;
operate the camera, subsequent to acquiring the second image, to acquire further images of the reflective elements reflective elements of the first identifying marker and the second identifying marker and to calculate respective further positions of the first identifying marker in response to the further images;
filter the further positions in response to a difference between the second position and the first position; and
in response to the filtered further positions, generate the virtual augmented reality images of at least the portion of the instrument or tool on the display of the head-mounted device,
wherein when the difference is not greater than a threshold value, the filtering comprises applying filtering with a first cutoff frequency, and when the difference is greater than the threshold value, the filtering comprises filtering with a second cutoff frequency greater than the first cutoff frequency.

29. (canceled)

30. (canceled)

31. (canceled)

32. The system of claim 28, wherein the first and the second positions are two-dimensional positions measured in pixels, and wherein the difference between the second position and the first position comprises a velocity from the first position to the second position measured in pixels per second.

33. The system of claim 28, wherein the first and the second positions are three-dimensional positions, and wherein the difference between the second position and the first position comprises a velocity from the first position to the second position.

34. (canceled)

35. (canceled)

36. (canceled)

37. (canceled)

38. The system of claim 28, wherein the second cutoff frequency is automatically adjusted over time by the one or more processors.

39. The system of claim 38, wherein the first cutoff frequency is automatically adjusted over time by the one or more processors.

40. The system of claim 28, wherein the filtering comprises use of one or more low-pass filters.

41-136. (canceled)
